(19) United States
(12) Patent Application Publication
(10) Pub. No.: US 2022/0125550 A1
(43) Pub. Date: Apr. 28, 2022

(54) SUPPORTER FOR MEDICAL CAMERA

(71) Applicant: 3D MediVision Inc., Seoul (KR)

(72) Inventors: Kijin KIM, Seongnam-si (KR); Taehwan KIM, Seoul (KR); Insu HAN, Suwon-si (KR)

(73) Assignee: 3D MediVision Inc., Seoul (KR)

(21) Appl. No.: 17/566,484

(22) Filed: Dec. 30, 2021

Related U.S. Application Data
(63) Continuation of application No. PCT/KR2019/018155, filed on Dec. 20, 2019.

(30) Foreign Application Priority Data
Dec. 16, 2019 (KR) 10-2019-0168193

(51) Int. Cl. A61B 90/50; A61B 90/00

(52) U.S. Cl. CPC A61B 90/50; A61B 90/06; A61B 90/361

(57) ABSTRACT

A supporter in which a position of a camera configured to capture an image of a surgical operation is controlled, includes: a body; a first arm coupled to the body; a second arm coupled to the first arm; a first joint coupled to the body and the first arm; a second joint coupled to the first arm and the second arm; a third joint coupled to the second arm and the camera; and a controller configured to control the first joint, the second joint and the third joint to change the position of the camera.

### CROSS-REFERENCE TO RELATED APPLICATIONS

This application is a Continuation of International Application No. PCT/KR2019/018155 filed Dec. 20, 2019, which claims benefit of priority to Korean Patent Application No. 10-2019-0168193 filed Dec. 16, 2019, the entire contents of which are incorporated herein by reference.

### TECHNICAL FIELD

The present disclosure relates to a supporter for a medical camera, and more particularly, to a supporter in which a position of a camera configured to capture an image of a surgical operation may be controlled.

### BACKGROUND

In general, a camera for capturing an image of a surgical site during a surgical operation is fixed to a supporter, such as a tripod. In order to optimally capture the image of the surgical site, the camera is required to be installed at a distance of about 40 cm from the surgical site. As such, the camera may be positioned near the head of the surgeon who performs the surgical operation. This may cause interference between the head of the surgeon and the camera, such as collision of the head of the surgeon with the camera during the surgical operation.

To avoid such a situation, the camera may be fixed at a position higher than the head of the surgeon. In this case, the surgical site may be blocked by the head of the surgeon, which makes it difficult to capture the surgical site.

### SUMMARY

An aspect provides a supporter for a medical camera which is capable of automatically controlling a position of the camera in real time such that the camera that captures a surgical operation avoids the human body of a surgeon and thus an image of a surgical site does not overlap the human body of the surgeon.

According to an example embodiment of the present disclosure, there is a supporter for controlling a position of a camera configured to capture a surgical operation, may include: a body; a first arm coupled to the body; a second arm coupled to the first arm; a first joint coupled to the body and the first arm; a second joint coupled to the first arm and the second arm; a third joint coupled to the second arm and the camera; and a controller configured to control the first joint, the second joint and the third joint to change the position of the camera. The camera may be configured to capture the surgical operation to produce a surgical operation video, and the controller may be configured to control the first joint, the second joint and the third joint such that an image of a surgical site in the surgical operation video does not overlap a portion of a human body of a surgeon.

In an example embodiment, the controller may be configured to further identify the surgical site in the surgical operation video, and control the first joint, the second joint and the third joint such that a focus corresponding to a center of the identified surgical site corresponds to a center of an angle of view of the camera.

In an example embodiment, the first joint may be configured to connect the body and a first end of the first arm, and the controller may be configured to further control a horizontal position of the first arm such that the camera moves on an arc centered at the first end of the first arm.

In an example embodiment, the controller may be configured to further control a vertical position of the first arm such that the camera moves vertically with respect to the first end of the first arm.

In an example embodiment, the second joint may be configured to connect a second end of the first arm and a first end of the second arm, and the controller may be configured to further control the second joint such that the camera moves in a conical shape centered at the first end of the second arm.

In an example embodiment, the controller may be configured to further control the second joint such that the camera rotates in a certain direction with respect to the first end of the second arm.

In an example embodiment, the controller may be configured to further control the second joint such that the second arm is positioned at a certain angle from a central line including the first end of the second arm.

In an example embodiment, the third joint may include a plurality of sensors configured to measure a distance between a portion of the human body of the surgeon and the third joint, and the controller may be configured to further estimate a position of a head of the surgeon using the measured distance and control the first joint, the second joint and the third joint such that the head does not overlap the image of the surgical site in the surgical operation video using the estimated position of the head.

In an example embodiment, the camera may include a lens, a horizontal rotation part configured to horizontally rotate the lens, and a vertical rotation part configured to vertically rotate the lens. The controller may be configured to further control the horizontal rotation part and the vertical rotation part such that the image of the surgical site in the surgical operation video and the portion of the human body of the surgeon do not overlap each other.

In an example embodiment, the controller may be configured to further control the horizontal rotation part and the vertical rotation part such that the focus corresponding to the center of the identified surgical site corresponds to the center of the angle of view of the camera.

Further, according to another example embodiment of the present disclosure, there is provided a supporter including: a body; a first arm coupled to the body; a second arm coupled to the first arm and configured to support a camera that captures a surgical operation to produce a surgical operation video; at least one sensor provided close to the camera and configured to measure a distance between the camera and a head of an surgeon who performs the surgical operation; and a controller configured to trace a position of the head in real time using the distance measured by the sensor, move the camera based on the traced position of the head and a preset avoidance operation distance, and control the first arm and the second arm such that a center of an angle of view of the moved camera is aligned with a center of a surgical site in the surgical operation video.

In an example embodiment, when a distance between the camera and the head corresponds to the preset avoidance operation distance, the controller may be configured to further control the first arm and the second arm such that the camera moves in a direction opposite to the position of the head.

In an example embodiment, the controller may be configured to further control a horizontal position of the first arm such that the camera moves on an arc centered at one end of the first arm.

In an example embodiment, the controller may be configured to further control a vertical position of the first arm such that the camera moves vertically with respect to one end of the first arm.

In an example embodiment, the controller may be configured to further control the second arm such that the camera rotates in a certain direction with respect to one end of the second arm.

In an example embodiment, the preset avoidance operation distance may be 30 mm.

The technology disclosed herein may have the following effects. However, specific example embodiments are not meant to provide all of the following effects or merely the following effects. Thus, it is not to be understood that the scope of the technology disclosed herein is limited to the specific example embodiments.

A supporter for a medical camera according to an example embodiment of the present disclosure is capable of avoiding collision of a human body of a surgeon with a camera that captures a surgical operation and automatically controlling a position of the camera in real time such that an image of a surgical site does not overlap the human body of the surgeon.

### BRIEF DESCRIPTION OF THE DRAWINGS

FIG. 1 is a diagram illustrating a supporter according to an example embodiment of the present disclosure.

FIGS. 2A and 2B are diagrams illustrated to explain an installation position of a sensor illustrated in FIG. 1.

FIG. 3 is a diagram illustrating a detailed configuration of a controller illustrated in FIG. 1.

FIGS. 4A and 4B are diagrams illustrated to explain a rotational operation of a first joint illustrated in FIG. 1.

FIGS. 5A and 5B are diagrams illustrated to explain a rotational operation of a second joint illustrated in FIG. 1.

FIG. 6 is a diagram illustrated to explain a rotational operation of a third joint illustrated in FIG. 1.

FIG. 7 is a diagram illustrated to explain an avoidance operation of the supporter according to an example embodiment of the present disclosure.

### DETAILED DESCRIPTION

Descriptions of embodiments of the present disclosure are merely structural to functional descriptions of the present disclosure. Thus, the scope of the present disclosure should not be interpreted as being limited to example embodiments described herein. That is, the example embodiments may be varied in various forms. Thus, the scope of the present disclosure should be understood as including equivalents by which the technical sprit may be implemented. Further, all of the purposes or the effects disclosed herein are not meant to be provided by specific example embodiments. Thus, it is not to be understood that the scope of the present disclosure is limited to the specific example embodiments.

Further, terms used herein are to be understood as follows.

Terms “first,” “second,” and the like are used to distinguish one constituent element from another constituent element, and the scope of the present disclosure is not limited by these terms. For example, the first constituent element may be referred to as the second constituent element, and conversely, the second constituent element may be referred to as the first constituent element

When one constituent element is referred to as being “connected” to another constituent element, the one constituent element may be directly connected to another constituent element, or may be connected to another constituent element by intervening yet another constituent element therebetween. On the contrary, when one constituent element is said to be “directly connected” to another constituent element, it should be understood that there is no other constituent element between these constituent elements. Further, various expressions used when explaining a relationship between constituent elements, such as “between” and “just between” or “adjacent to” and “directly adjacent to” and the like, should be interpreted as well.

Expressions in the singular form should be understood to encompass expressions in the plural form unless the context clearly indicates otherwise. The term “includes”, “has” or the like are intended to include features, numeric characters, steps, operations, constituent elements, parts, or a combination thereof, and should be understood not to exclude one or more other features, numeric characters, steps, operations, constituent elements, parts, or a combination thereof, or additional features and the like.

Reference numerals (for example, a, b, c, and the like) used in respective steps are used for the sake of convenience in description and do not state the order of the respective steps. Respective steps may be performed in an order different from the stated order unless the context clearly dictates a specific order. That is, respective steps may be performed in the stated order and may be performed substantially simultaneously and vice versa.

The present disclosure may be implemented with computer-readable codes in a computer-readable recording medium. The computer-readable recording medium is any data recording device capable of storing data that may be read by a computer system. Examples of the computer-readable recording medium may include a read only memory (ROM), a random access memory (RAM), compact disk (CD)-ROMs, magnetic tapes, floppy disks, optical data storage devices, and the like. Further, the computer readable recording medium may be distributed through network-connected computer systems. Thus, the computer-readable codes are stored and executed in a distributed manner.

Unless otherwise defined, all terms used herein have the same meaning commonly understood by those skilled in the art to which the present disclosure pertains. The commonly-used predefined terms should be interpreted as consistent with the meanings of the context in the related art and should not be interpreted as having ideal or excessive formal meanings unless otherwise defined in this application.

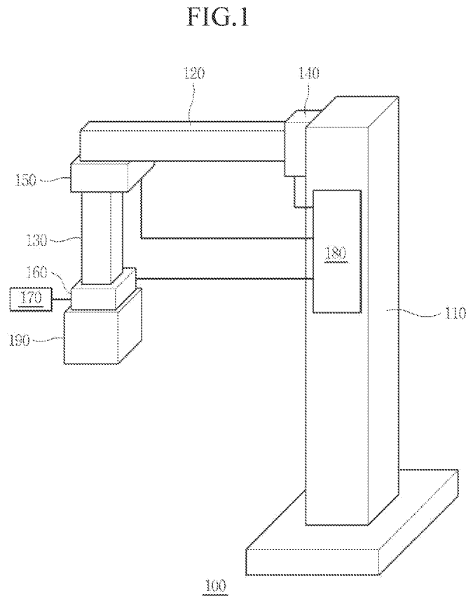

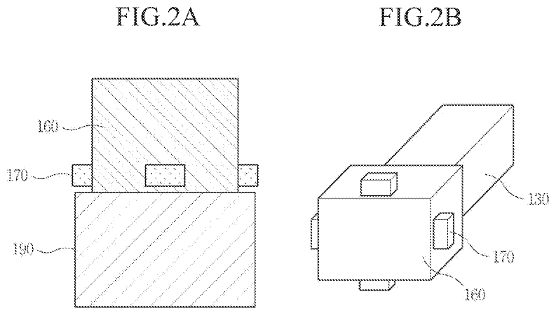

FIG. 1 is a diagram illustrating a supporter according to an example embodiment of the present disclosure. FIGS. 2A and 2B are diagrams illustrated to explain an installation position of a sensor illustrated in FIG. 1.

Referring to FIG. 1, a supporter 100 according to an example embodiment of the present disclosure supports a camera 190 for capturing a surgical operation to produce a surgical operation video, and controls a position of the camera 190. The supporter 100 includes a body 110, a first arm 120, a second arm 130, a first joint 140, a second joint 150, a third joint 160, a sensor 170, and a controller 180.

The body 110 is a base support pedestal which is installed at a certain height from the ground or an operating table to support the first arm 120 and the second arm 130. The body 110 has a certain length in a vertical direction from the ground. The body 110 may have an internal space in which a connection cable that connects the first joint 140, the second joint 150, the third joint 160, the sensor 170 and the controller 180, and a module that constitutes the controller 180 may be arranged. Further, the body 110 may include an external display module (not illustrated) which displays the surgical operation video obtained by the camera 190 thereon.

The first arm 120 is an intermediate support pedestal coupled to the body 110 to support the second arm 130. In an example embodiment, the first arm 120 is coupled to an upper end of the body 110 via the first joint 140, and has a certain length in a direction perpendicular to a longitudinal direction of the body 110.

The second arm 130 is a lower support pedestal coupled to the first arm 120 to support the camera 190. In an example embodiment, the second arm 130 is coupled to the first arm 120 via the second joint 150 and is coupled to the camera 190 via the third joint 160.

The first joint 140 connects the body 110 and one end of the first arm 120. The first joint 140 is controlled by the controller 180 and rotates a central axis of the first arm 120 at a certain angle with respect to one of an up-down direction and a left-right direction.

That is, the first joint 140 rotates the first arm 120 in a horizontal direction to primarily adjust the position of the camera 190, and rotates the first arm 120 in a vertical direction to adjust the height of the camera 190.

The second joint 150 connects the other end of the first arm 120 and one end of the second arm 130. The second joint 150 is controlled by the controller 180 and rotates a center axis of the second arm 130 at a certain angle with respect to one of a front-rear direction and the left-right direction. That is, the second joint 150 rotates the second arm 130 in all directions to secondary adjust the position of the camera 190.

The third joint 160 connects the other end of the second arm 130 and the camera 190. The third joint 160 is controlled by the controller 180 and rotates the camera 190 at a certain angle with respect to the front-rear direction and the left-right direction. That is, the third joint 160 adjusts an angle of view of the camera 190.

The sensor 170 is disposed in the third joint 160 and measures a distance between the third joint 160 and a surgeon. The sensor 170 sends information about the measured distance to the controller 180.

The sensor 170 may be installed at a location close to the camera 190, for example, in a lower end portion or lower surface of the third joint 160 as illustrated in FIGS. 2A and 2B, to measure a distance between the camera 190 and a portion of the human body of the surgeon.

Further, a plurality of sensors 170 may be provided. For example, at least four of sensors 170 may be provided in the third joint 160 in all directions.

The sensor 170 may include at least one of an IR (infrared ray) sensor, a PSD (position sensitive device) sensor, a laser sensor and a supersonic sensor, and may use other sensors configured to sense a distance.

The controller 180 controls the first joint 140, the second joint 150 and the third joint 160 using the distance between the third joint 160 and the portion of the human body of the surgeon, which is measured by the sensor 170, and the surgical operation video received from the camera 190.

The controller 180 identifies a surgical site from the surgical operation video received from the camera 190, and controls the first joint 140, the second joint 150 and the third joint 160 such that a focus corresponding to the center of the identified surgical site corresponds to the center of the angle of view of the camera 190.

Further, the controller 180 estimates a portion of the head of the surgeon using the distance between the third joint 160 and the portion of the human body of the surgeon, which is measured by the sensor 170, and identifies the surgical site from the surgical operation video received from the camera 190. The controller 180 controls the first joint 140, the second joint 150 and the third joint 160 such that an image of the identified surgical site and the head of the surgeon do not overlap each other.

The controller 180 determines whether or not the distance between the third joint 160 and the head of the surgeon corresponds to a preset avoidance operation distance. When it is determined that the distance between the third joint 160 and the head of the surgeon corresponds to the preset avoidance operation distance, the controller 180 rotates the second joint 150 and the third joint 160 by a certain angle in a direction opposite to the position of the head of the surgeon, thus changing the position of the camera 190. In an example embodiment of the present disclosure, the preset avoidance operation distance may be about 30 mm, but is not limited thereto. The avoidance operation distance may be adjusted in a manual manner.

In an example embodiment, the controller 180 may compute the rotational angles of the second joint 150 and the third joint 160 based on the length of the second arm 130 and the position of the head of the surgeon. That is, the controller 180 may control the second joint 150 and the third joint 160 to allow the camera 190 to capture an image of the surgical site without interference such as hitting the head of the surgeon.

At least one of camera 190 may be installed in an operating room to acquire a surgical operation video by capturing a surgical field. The at least one of camera 190 transmits the acquired surgical operation video to the controller 180. The at least one of camera 190 may be configured with a two-dimensional (2D) camera that acquires a 2D image or a three-dimensional (3D) camera that acquires a 3D image.

In an example embodiment, when the camera 190 is the 2D camera, the camera 190 may convert a 2D image into a 3D image and transmit the same to the controller 180. Further, the camera 190 may be configured with a stereo camera, a high-definition (HD) pan-tilt-zoom (PTZ) camera that supports pan-tilt and zoom functions, or the like, which is capable of capturing the surgical field.

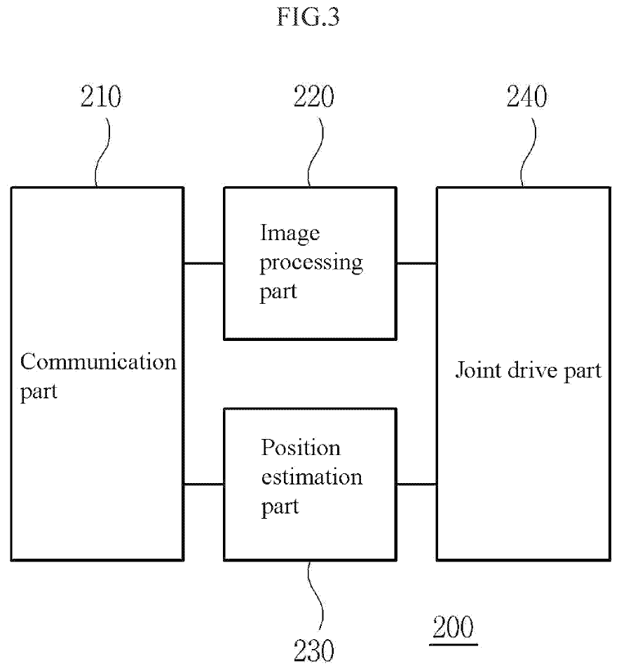

FIG. 3 is a diagram illustrating a detailed configuration of the controller illustrated in FIG. 1.

Referring to FIG. 3, the controller 180 includes a communication part 210, an image processing part 220, a position estimation part 230, and a joint drive part 240. The communication part 210 communicates with the sensor 170 and the camera 190 to receive information about the distance between the third joint 160 and the portion of the human body of the surgeon and the surgical operation video. The communication part 210 may communicate with the sensor 170 and the camera 190 via a data communication network such as a wide area network (WAN), a mobile radio communication network, a wired communication network, etc.

The image processing part 220 receives the surgical operation video via the communication part 210 and identifies a surgical site from the received surgical operation video. For example, the image processing part 220 may identify the surgical site included in each of a plurality of image frames of the surgical operation video based on a previously-learned anatomical model. In an example embodiment, the anatomical model may be implemented by processing images of recorded surgical sites using an image processing technique such as CT (Computed Tomography), MRI (Magnetic Resonance Imaging), fluoroscopy, temperature recording, ultrasound, OCT (Optical Coherence Tomography), DOT (Diffused Optical Tomography), thermal capturing, impedance capturing, laser capturing, nanotube X-ray capturing, or the like. The anatomical model may be learned by a patient, or with a standard human body or the like, and may also be learned with deformable anatomic postures.

The present disclosure is not limited to the example embodiment described above. In another example embodiment, at least one identifiable recognition marker may be displayed on a surgical site, and the image processing part 220 may detect the recognition marker in the surgical operation video and identify the surgical site along an outline of the recognition marker using the image processing technique.

The position estimation part 230 receives the information about the distance between the third joint 160 and the portion of the human body of the surgeon through the communication part 210, and estimates the position of the head of the surgeon using the information about the distance between the third joint 160 and the portion of the human body of the surgeon. For example, the position estimation part 230 may model a human body surface of the surgeon with reference to distances between the third joint 160 and some protruded points of the human body of the surgeon, which are close to the third joint 160, to estimate the position of the head of the surgeon.

The joint drive part 240 controls the first joint 140, the second joint 150 and the third joint 160 such that a focus corresponding to the center of the surgical site in the surgical operation video corresponds to the center of the angle of view of the camera 190. For example, the joint drive part 240 may recognize coordinates of the center point of the identified surgical site and control the first joint 140, the second joint 150 and the third joint 160 such that the recognized coordinates correspond to the center point of a screen of the camera 190.

Further, the joint drive part 240 determines, in real time, whether or not the distance between the third joint 160 and the head of the surgeon corresponds to the avoidance operation distance. When it is determined that the distance between the third joint 160 and the head of the surgeon corresponds to the avoidance operation distance, the joint drive part 240 controls the second joint 150 and the third joint 160 to move the camera 190 in a direction opposite to the position of the head of the surgeon.

That is, the joint drive part 240 controls the first joint 140, the second joint 150 and the third joint 160 such that the camera 190 may accurately focus and continuously capture an image of the surgical site while automatically avoiding the head of the surgeon in real time. This makes it possible to prevent occurrence of interference between the camera 190 and the head of the surgeon, and acquire the image of the surgical site with a high degree of accuracy.

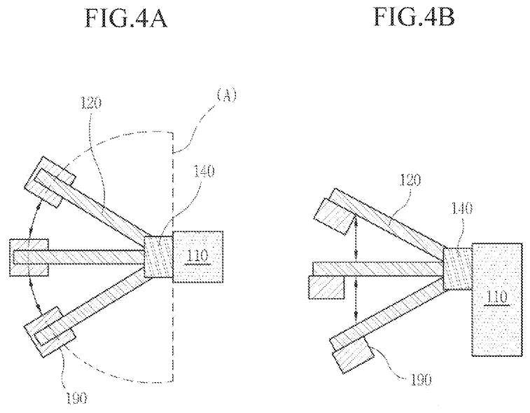

FIGS. 4A and 4B are diagrams illustrated to explain the rotational operation of the first joint illustrated in FIG. 1, FIG. 4A being a top view, and FIG. 4B being a side view.

Referring to FIGS. 4A and 4B, the first joint 140 is controlled by the controller 180 and rotates the first arm 120 in the horizontal direction and the vertical direction. The first joint 140 adjusts a horizontal position of the first arm 120 when the first arm 120 rotates horizontally to move the camera 190 by a certain angle on an arc A centered at one end of the first arm 120, as indicated by the arrows in FIG. 4A.

The first joint 140 adjusts a vertical position of the first arm 120 when the first arm 120 rotates vertically to move the camera 190 by a certain angle at a certain vertical height with respect to one end of the first arm 120, as indicated by the arrows in FIG. 4B.

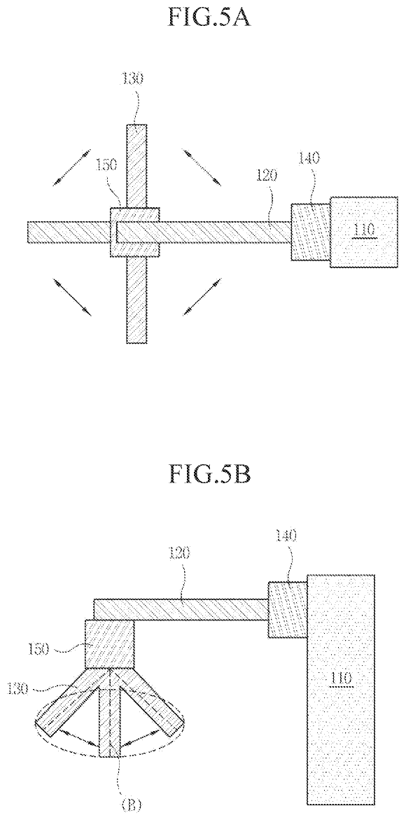

FIGS. 5A and 5B are diagrams illustrated to explain a rotational operation of the second joint illustrated in FIG. 1, FIG. 5A being a top view, and FIG. 5B being a side view.

Referring to FIGS. 5A and 5B, the second joint 150 is controlled by the controller 180 and rotates the second arm 130 in the front-rear direction and the left-right direction. The second joint 150 adjusts a position of the second arm 130 to rotate the camera 190 in a certain direction with respect to one end of the second arm 130, as indicated by the arrows in FIG. 5A.

The second joint 150 adjusts the position of the second arm 130 to move the camera 190 in a conical shape with respect to one end of the second arm 130, as indicated by the arrows in FIG. 5B. That is, the second joint 150 adjusts the position of the second arm 130 such that the second arm 130 is positioned at a certain angle from a central line B including one end of the second arm 130.

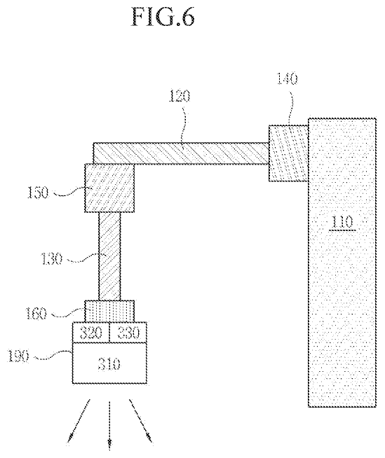

FIG. 6 is a diagram illustrated to explain a rotational operation of the third joint illustrated in FIG. 1.

Referring to FIG. 6, the third joint 160 performs a pan-tilt function to control the angle of view of the camera 190. The camera 190 according to an example embodiment of the present disclosure may include a lens 310, a horizontal rotation part 320, and a vertical rotation part 330. In an example embodiment, the horizontal rotation part 320 is coupled to the third joint 160 to horizontally rotate the lens 310. The vertical rotation part 330 is coupled to the third joint 160 to vertically rotate the lens 310.

The third joint 160 adjusts operations of the horizontal rotation part 320 and the vertical rotation part 330 under the control of the controller 180 such that the surgical site in the surgical operation video does not overlap an obstacle. Further, the third joint 160 adjusts operations of the horizontal rotation part 320 and the vertical rotation part 330 under the control of the controller 180 such that a focus corresponding to the center of a surgical site S corresponds to the center of the angle of view of the camera 190.

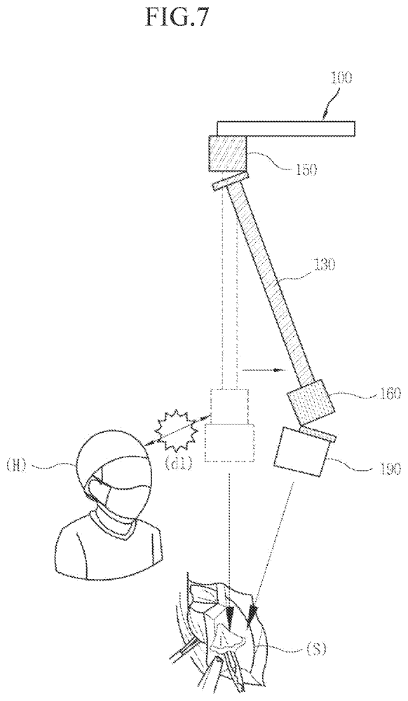

FIG. 7 is a diagram illustrated to explain the avoidance operation of the supporter according to an example embodiment of the present disclosure.

Referring to FIG. 7, the supporter 100 according to an example embodiment of the present disclosure senses a distance dl between the third joint 160 and the head H of the surgeon, and determines whether or not the sensed distance dl corresponds to the avoidance operation distance. Based on the determination result, the supporter 100 automatically performs the avoidance operation. That is, when it is determined that the distance dl between the third joint 160 and the head H of the surgeon corresponds to the preset avoidance operation distance, the supporter 100 automatically controls the second joint 150 and the third joint 160 to move the camera 190 in a direction opposite to the position of the head H of the surgeon.

In this case, the second joint 150 and the third joint 160 are controlled such that the focus corresponding to the center of the surgical site S corresponds to the center of the angle of view of the camera 190. Therefore, it is possible to capture an image of the surgical site S with high precision in focus without interference with the camera 190 such as hitting the head H of the surgeon in a state in which the head H of the surgeon does not overlap the image of the surgical site S.

While the foregoing has been described with reference to preferred example embodiments of the present disclosure, it will be appreciated by those skilled in the art that various modifications and variations may be made to the present disclosure without departing from the spirit and scope of the present disclosure as set forth in the following claims.

What is claimed is:

1. A supporter in which a position of a camera configured to capture an image of a surgical operation is controlled, comprising:
a body;
a first arm coupled to the body;
a second arm coupled to the first arm;
a first joint coupled to the body and the first arm;
a second joint coupled to the first arm and the second arm;
a third joint coupled to the second arm and the camera; and
a controller configured to control the first joint, the second joint and the third joint to change the position of the camera,
wherein the camera is configured to capture the surgical operation to produce a surgical operation video, and
the controller is configured to control the first joint, the second joint and the third joint such that an image of a surgical site in the surgical operation video does not overlap a portion of a human body of a surgeon.

2. The supporter of claim 1, wherein the controller is configured to further identify the surgical site in the surgical operation video, and control the first joint, the second joint and the third joint such that a focus corresponding to a center of the identified surgical site corresponds to a center of an angle of view of the camera.

3. The supporter of claim 2, wherein the first joint is configured to connect the body and a first end of the first arm, and
the controller is configured to further control a horizontal position of the first arm such that the camera moves on an arc centered at the first end of the first arm.

4. The supporter of claim 3, wherein the controller is configured to further control a vertical position of the first arm such that the camera moves vertically with respect to the first end of the first arm.

5. The supporter of claim 4, wherein the second joint is configured to connect a second end of the first arm and a first end of the second arm, and
the controller is configured to further control the second joint such that the camera moves in a conical shape centered at the first end of the second arm.

6. The supporter of claim 5, wherein the controller is configured to further control the second joint such that the camera rotates in a certain direction with respect to the first end of the second arm.

7. The supporter of claim 6, wherein the controller is configured to further control the second joint such that the second arm is positioned at a certain angle from a central line including the first end of the second arm.

8. The supporter of claim 7, wherein the third joint includes a plurality of sensors configured to measure a distance between a portion of the human body of the surgeon and the third joint, and
the controller is configured to further estimate a position of a head of the surgeon using the measured distance and control the first joint, the second joint and the third joint such that the head does not overlap the image of the surgical site in the surgical operation video using the estimated position of the head.

9. The supporter of claim 8, wherein the camera includes:
a lens;
a horizontal rotation part configured to horizontally rotate the lens; and a vertical rotation part configured to vertically rotate the lens,
wherein the controller is configured to further control the horizontal rotation part and the vertical rotation part such that the image of the surgical site in the surgical operation video and the portion of the human body of the surgeon do not overlap each other.

10. The supporter of claim 9, wherein the controller is configured to further control the horizontal rotation part and the vertical rotation part such that the focus corresponding to the center of the identified surgical site corresponds to the center of the angle of view of the camera.

11. A supporter comprising:
a body;
a first arm coupled to the body;
a second arm coupled to the first arm and configured to support a camera that captures a surgical operation to produce a surgical operation video;
at least one sensor provided close to the camera and configured to measure a distance between the camera and a head of a surgeon who performs the surgical operation; and
a controller configured to trace a position of the head in real time using the distance measured by the sensor, move the camera based on the traced position of the head and a preset avoidance operation distance, and control the first arm and the second arm such that a center of an angle of view of the moved camera is aligned with a center of a surgical site in the surgical operation video.

12. The supporter of claim 11, wherein, when a distance between the camera and the head corresponds to the preset avoidance operation distance, the controller is configured to further control the first arm and the second arm such that the camera moves in a direction opposite to the position of the head.

13. The supporter of claim 11, wherein the controller is configured to further control a horizontal position of the first arm such that the camera moves on an arc centered at one end of the first arm.

14. The supporter of claim 11, wherein the controller is configured to further control a vertical position of the first arm such that the camera moves vertically with respect to one end of the first arm.

15. The supporter of claim 11, wherein the controller is configured to further control the second arm such that the camera rotates in a certain direction with respect to one end of the second arm.

16. The supporter of claim 11, wherein the preset avoidance operation distance is 30 mm.
